(19) United States
(12) Patent Application Publication
(10) Pub. No.: US 2013/0267590 A1
(43) Pub. Date: Oct. 10, 2013

(54) RETIGABINE COMPOSITIONS

(71) Applicants: Snehalatha Movva, Hyderabad (IN); Mary Tom Teenu, Calicut (IN); Badri Narayanan Vishwanathan, Chennai (IN)

(72) Inventors: Snehalatha Movva, Hyderabad (IN); Mary Tom Teenu, Calicut (IN); Badri Narayanan Vishwanathan, Chennai (IN)

(73) Assignee: Dr. Reddy's Laboratories Limited, Hyderabad (IN)

(21) Appl. No.: 13/613,649

(22) Filed: Sep. 13, 2012

Related U.S. Application Data
(60) Provisional application No. 61/551,636, filed on Oct. 26, 2011.

(30) Foreign Application Priority Data
Sep. 13, 2011 (IN) 3137/CHE/2011

(51) Int. Cl. A61K 31/27

(52) U.S. Cl. CPC A61K 31/27
USPC 514/485

(57) ABSTRACT

Stable premixes comprising retigabine, or pharmaceutically acceptable salts thereof, and at least one pharmaceutically acceptable carrier, and process for preparing the same are disclosed. Stable premixes comprising retigabine, or pharmaceutically acceptable salts thereof, and at least one pharmaceutically acceptable inorganic carrier, and processes for preparing the same and incorporating them in compositions that are employed for therapeutic uses and methods of treatment.

This application claims priority to Indian Application 3137/CHE/2011, filed on Sep. 13, 2011; U.S. Provisional Application No. 61/551,636, filed on Oct. 26, 2011; all of which are hereby incorporated by reference in their entirety.

### BACKGROUND

Aspects of the present application relate to retigabine premixes with pharmaceutical carriers, pharmaceutical compositions containing the premixes, and processes for preparing the same. In particular aspects, the present application relates to stable premixes comprising retigabine, or pharmaceutically acceptable salts thereof, and at least one pharmaceutically acceptable carrier, and process for preparing the same. Particular aspects relate to stable premixes comprising retigabine, or pharmaceutically acceptable salts thereof, and at least one pharmaceutically acceptable inorganic carrier, and processes for preparing the same. In aspects, the application further relates to therapeutic uses and methods of treatment employing the compositions.

Retigabine (Formula I), referred to as “ezogabine” in the United States and having chemical names: ethyl 2-amino-4-(4-fluorobenzylamino) phenylcarbamate; or N-[2-amino-4-(4-fluorobenzylamino)-phenyl]carbamic acid ethyl ester; is a pharmaceutical substance with anticonvulsive, antipyretic, and analgesic activity, and is used in pharmaceutical preparations. It has an empirical formula of C₁₆H₁₈FN₃O₂ and a molecular weight of 303.33.

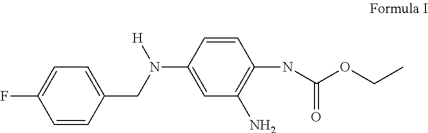 [SMILES: CCOC(=O)Nc1ccc(NCc2ccc(F)cc2)cc1N]

Ezogabine was approved for use in the U.S.A. in 2011 and is available in products sold as POTIGA® film-coated immediate release tablets having 50, 200, 300, and 400 mg strengths. It is indicated for adjunctive treatment of partial-onset seizures in patients aged 18 years and older. The inactive excipients of POTIGA® include carmine (50 and 400 mg tablets), croscarmellose sodium, FD&C Blue No. 2 (50, 300, and 400 mg tablets), hypromellose, iron oxide yellow (200 and 300 mg tablets), magnesium stearate, microcrystalline cellulose, polyethylene glycol 3350, polyvinyl alcohol, talc, and titanium dioxide. Also in 2011, the drug was approved by the European Commission for adjunctive therapy of adult epilepsy patients with partial onset seizures. Retigabine is an ingredient in products sold in Europe as TROBALT®.

Epilepsy is a condition in which a person has recurrent seizures. A seizure is defined as an abnormal, disorderly discharging of the brain's nerve cells, resulting in a temporary disturbance of motor, sensory, or mental function. There are many types of seizures, depending primarily on what part of the brain is involved. The exact mechanism of action for retigabine in treating seizure is not known. However in vitro studies indicate that the drug enhances transmembrane potassium currents mediated by the KCNQ (Kv7.2 to 7.5) family of ion channels. By activating KCNQ channels, retigabine is thought to stabilize the resting membrane potential and reduce brain excitability. In vitro studies suggest that ezogabine may also exert therapeutic effects through augmentation of GABA-mediated currents.

U.S. Pat. No. 5,384,330 discloses retigabine and salts thereof. It discloses the preparation of retigabine and retigabine dihydrochloride. It further discloses method of producing an anti-epileptic, muscle relaxing, fever reducing, peripherally analgesic or anticonvulsive effect in a patient by administering effective amount of retigabine. Crystalline modifications A, B, and C of retigabine and processes for preparing the same have been disclosed in U.S. Pat. No. 6,538,151.

Tendency of the polymorphic forms to convert into other crystalline, polymorphic forms, and the fact that the crystalline forms have different solubility profiles, is disclosed in International Application Publication No. WO 98/31663. The differences in the solubility profiles of the different crystalline forms may lead to an undesired, irregular uptake of the active ingredient, following administration of a dose.

International Application Publication No. WO 2002/080898 discloses formulating crystalline retigabine in the form of hard gelatin capsules containing 50, 100, and 200 mg of the active ingredient. It further discloses that the granules can be subjected to coating, encapsulation, or compressed into tablet dosage form.

G. Blackburn-Munro et al., “Retigabine: Chemical Synthesis to Clinical Application,” CNS Drug Reviews, Vol. 11, No. 1, pp. 1-20, 2005 disclose the dihydrochloride salt of retigabine as being hygroscopic and unstable under medium to long-term storage at −18° C. It also mentions that retigabine is preferably stored as the free base, isolated from light. It discloses the tendency of retigabine to oxidize upon contact with air.

In addition, retigabine formulations are described in International Application Publication No. WO 01/66081A2, prepared using a melt granulation technique to obtain a matrix composition consisting of only crystalline retigabine and sucrose fatty acid ester. However large quantities of sucrose fatty acid ester often lead to undesired results due to its emulsifying effect. Furthermore, the recommended formulations are related to delayed release.

International Application Publication No. WO 2009/118167 relates to pharmaceutical formulations containing solid dispersions of amorphous form drugs with polymeric materials, additionally containing polymeric solutions as stabilizers. It discloses dosage forms such as pellets, tablets, capsules, etc.

International Application Publication No. WO 2010/105823 discloses an intermediate comprising retigabine in a solid, non-crystalline form (solid solution or amorphous) and a surface stabilizator. It also claims a process of selecting a suitable excipient as a surface stabilizer, which adds to the complexity of the process. It discloses immediate release and modified release formulations containing retigabine intermediate.

International Application Publication No. WO 2011/039369 discloses a stable amorphous solid mixture of retigabine and at least one pharmaceutically acceptable carrier, wherein the carriers are polymeric substances, processes for preparing the same, solid pharmaceutical formulations comprising the same, and mixtures or formulations stabilized in a container or packaging. The publication discloses different techniques for preparation of the solid mixture, such as melt extrusion, lyophilization, freeze drying, etc., which are complex processes.

There remains a need for stable pharmaceutical compositions comprising retigabine or a pharmaceutically acceptable salt thereof. Further, there also exists a need for a simplified process for producing stable retigabine premixes with commonly available excipients along with improved flow properties and compressibility of premixes that can be easily formulated to give desired pharmaceutical compositions.

### SUMMARY

In aspects, the present application provides stable premix compositions comprising retigabine or a pharmaceutically acceptable salt thereof, pharmaceutical compositions comprising the premixes, and processes for preparing the same.

In embodiments, the application includes premixes for use in pharmaceutical formulations of retigabine, said premixes comprising a combination of: (a) retigabine or a pharmaceutically acceptable salt thereof; and (b) an inorganic carrier.

In an aspect, the application provides processes for preparing premixes for use in pharmaceutical formulations of retigabine, embodiments of the processes comprising: (a) dissolving retigabine or a pharmaceutically acceptable salt thereof in a solvent to form a solution; and (b) adsorbing the solution onto an inorganic carrier to obtain a premix.

In embodiments, the application relates to stable premixes comprising retigabine or pharmaceutically acceptable salt thereof and at least one pharmaceutically acceptable inorganic carrier, wherein the retigabine in a premix is in an amorphous form.

In embodiments, the application relates to stable premixes comprising retigabine or pharmaceutically acceptable salt thereof and at least one pharmaceutically acceptable inorganic carrier, wherein the retigabine in a premix is in the form of a solid dispersion.

In embodiments, the application relates to stable premixes comprising retigabine or pharmaceutically acceptable salt thereof and at least one pharmaceutically acceptable inorganic carrier, wherein the retigabine in a premix is in the form of a solid solution.

In embodiments, the application relates to stable premixes of retigabine wherein weight ratios of retigabine to inorganic carrier are in the range of about 10:90 to about 90:10.

In embodiments, the application provides stable premixes and/or compositions of retigabine, which are substantially free of drug degradation impurities.

In embodiments, the application relates to stable compositions and/or formulations, wherein total drug-related impurities are less than about 5% of the label content of retigabine.

In embodiments, the application provides processes for producing stable premixes, wherein the water content of a premix as determined using a Karl Fischer technique is about 0.5-10% by weight.

In embodiments, the application provides processes for producing stable premixes and/or compositions, wherein a premix has losses on drying at 105° C. in the range of about 0.25-10%, or about 0.5-5%, by weight.

In embodiments, the present application provides premixes of retigabine having enhanced flowability and compressibility properties, and can be easily formulated into pharmaceutical formulations.

In embodiments, retigabine premixes of the present application have Carr index values in the range of about 1-40%.

In embodiments, a retigabine premix further comprises one or more additional pharmaceutically acceptable excipients.

In embodiments, premixes are used directly, or used in combination with additional excipients, to prepare desired pharmaceutical dosage forms. In an aspect, the application includes methods of preparing dosage forms containing retigabine premixes.

In an aspect, the application includes forms of packaging for the retigabine premixes and/or compositions, which provide stability during storage and transportation.

In an aspect, the application includes methods of treating patients suffering from conditions, e.g., epilepsy, as an adjunctive treatment of partial onset seizures with or without secondary generalisation in adults, using pharmaceutical formulations of the present application.

### BRIEF DESCRIPTION OF THE DRAWINGS

FIG. 1 shows comparative X-ray powder diffraction (XRPD) patterns for a premix prepared according to Example 1, where A represents the crystalline retigabine ingredient, B represents a premix, and C represents a similarly prepared placebo, omitting the retigabine.

FIG. 2 shows comparative XRPD patterns for a premix prepared according to Example 2, where A represents the crystalline retigabine ingredient, B represents a premix, and C represents a similarly prepared placebo, omitting the retigabine.

FIG. 3 shows XRPD patterns for a premix of Example 1, after storage at 2 to 8° C. for 16 months.

FIG. 4 shows XRPD patterns for a premix of Example 2, after storage at 2 to 8° C. for 16 months.

FIG. 5 shows XRPD patterns for a premix of Example 1, after storage at 2 to 8° C. for 16 months followed by storage at 40° C. and 75% relative humidity for 1 week.

FIG. 6 shows XRPD patterns for a premix of Example 2, after storage at 2 to 8° C. for 16 months followed by storage at 40° C. and 75% relative humidity for 1 week.

### DETAILED DESCRIPTION

Aspects of the present application relate to premix compositions comprising retigabine, pharmaceutical formulations containing the premixes, and processes for preparing the premixes and their pharmaceutical formulations.

As used herein the term “retigabine” includes the compound retigabine, pharmaceutically acceptable salts thereof, prodrugs thereof, the active metabolites of retigabine and the prodrugs thereof, including any of their polymorphs, solvates, and hydrates.

Pharmaceutically acceptable acid salts of retigabine include acid addition salts, formed from acids that provide non-toxic anions. Examples of pharmaceutically acceptable salts include but are not limited to, acetate, aspartate, benzoate, bicarbonate, carbonate, bisulfate, sulfate, chloride, bromide, benzene sulfonate, methyl sulfonate, phosphate, acid phosphate, lactate, maleate, malate, malonate, fumarate, lactate, tartrate, borate, camsylate, citrate, edisylate, esylate, formate, fumarate, gluceptate, glucuronate, gluconate oxalate, palmitate, pamoate, saccharate, stearate, succinate, tartrate, tosylate, and trifluoroacetate salts, and the like. The salts of the application may also be hemi-salts, including but not limited to a hemi-sulfate salt, and the like. The pharmaceutically acceptable acid addition salts of the compound of retigabine are prepared using methods well known in the art, for example by treating a solution or suspension of the free base with about one chemical equivalent of a pharmaceutically acceptable acid.

The term “premix” is used herein to describe combinations of retigabine, including any of its salts, etc., and at least one pharmaceutical carrier, wherein individual particles of the components cannot be distinguished using techniques such as optical microscopy. In embodiments, the drug is considered as being uniformly or non-uniformly distributed over surfaces of carrier particles. In other embodiments, the premixes are considered to be in the nature of molecular dispersions, or solid solutions. Simple mixtures of powdered ingredients will not constitute premixes. Some methods for preparing premixes are described herein.

The retigabine used for preparing the premix may be retigabine obtained by any methods, for example retigabine in a crystalline polymorphic form A, B, or C, or any mixture thereof. The crystalline polymorphs A, B, and C, or mixtures thereof, can be prepared by following any of the methods described in the art, such as the processes described in U.S. Pat. No. 6,538,151. Also, the retigabine can be directly used in a solution or dispersion as is obtained from a synthesis preparation.

In embodiments, the retigabine used in the preparation of premixes of the present application is a single crystalline polymorphic form such as form A, B, or C.

In other embodiments, the retigabine used in the preparation of premixes of the present application is a mixture of at least two of the crystalline polymorphic forms A, B, and C. In embodiments, retigabine used in the preparation of premixes of the present application is a mixture of at least two of the crystalline polymorphic forms A, B, and C, wherein the weight ratio of one polymorphic form to another is about 99:1 to about 1:99.

In embodiments, the retigabine used in the preparation of premixes of the present application is a mixture of crystalline polymorphic forms A and C, wherein the weight ratio of A:C is about 99:1 to about 1:99, for example, about 95:5.

The term “excipient” means a component of a pharmaceutical product that is not an active ingredient, such as a filler, diluent, carrier, etc. The excipients that are useful in preparing a pharmaceutical composition are generally safe, non-toxic and neither biologically nor otherwise undesirable, and are acceptable for veterinary use as well as human pharmaceutical use. “Pharmaceutically acceptable excipient” as used in the specification includes both one and more than one such excipient.

The term “formulation” refers to pharmaceutical dosage forms comprising premixes of retigabine. The pharmaceutical compositions of the present application can be prepared as solid oral dosage forms or liquid dosage forms. The solid forms include for example tablets, caplets, capsules (e.g., using hard or soft gelatin capsules), oral disintegrating dosage forms, chewable dosage forms, pills, granules, sachets, and the like. The liquid forms include for example solutions, syrups, suspensions or dispersions, emulsions like micro-emulsions or multiple-emulsions; elixirs, etc.

The term “optional” or “optionally” means that the subsequently described element, component, or circumstance may or may not be present, so that the description includes instances where the element, component, or circumstance occurs and instances where it does not.

The term “adsorption” used herein refers to the accumulation of particles (adsorbate) at a surface (adsorbent or substrate). It is a surface phenomenon wherein adsorbate does not penetrate the substrate. Adsorption may be achieved by various techniques, e.g., mixing, spraying, etc.

The premixes of the present application can be obtained by combining retigabine with a suitable inorganic carrier, in pharmaceutically acceptable proportions to yield desired characteristics of good stability and formulation properties.

The term “carrier” herein refers to any pharmaceutically acceptable inorganic material used as a substrate or adsorbent for the retigabine.

The term “solid dispersion” herein refers to solid products having at least two different components, such as a hydrophilic matrix and a hydrophobic drug. In some instances, the drug can be dispersed molecularly, in amorphous particles or in crystalline particles.

The term “stable” herein refers to chemical and polymorphic stability of premixes, wherein the retigabine premix of the application is stable toward moisture, heat, light, etc and does not convert into another polymorphic form or develop unacceptable impurity content during manufacturing and a subsequent storage period for commercially acceptable periods. Such periods may, for example, be about 6, 12, or 24 months, or any intermediate periods, during which a pharmaceutical product is kept in its original packaging at ambient temperatures.

In embodiments, the present application provides stable premixes and/or compositions of retigabine, which are substantially free of drug degradation impurities. The term “substantially free” means the presence of one or more drug degradation impurities in amounts less than about 5%, or about 4%, or about 3%, or about 2%, or about 1%, of the label content of retigabine.

In embodiments, the application relates to stable compositions and/or formulations wherein a total of drug-related impurities is less than about 5% of the label content of retigabine.

In embodiments, the application includes premixes for use in pharmaceutical formulations of retigabine, said premixes comprising in combination: (a) retigabine or a pharmaceutically acceptable salt thereof; and (b) an inorganic carrier.

In embodiments, the application provides processes for preparing premixes for use in pharmaceutical formulations of retigabine, embodiments of the processes comprising: (a) dissolving retigabine or a pharmaceutically acceptable salt thereof in a solvent to form a solution; (b) adsorbing the solution onto an inorganic carrier; and (c) optionally, drying the premix.

In embodiments, the application provides processes for preparing premixes for use in pharmaceutical formulations of retigabine, embodiments of the processes comprising: (a) dissolving retigabine or a pharmaceutically acceptable salt thereof in a solvent to form a solution; (b) mixing the resulting solution with inorganic carrier; and (c) optionally drying the premix.

In embodiments, the application provides processes for preparing premixes for use in pharmaceutical formulations of retigabine, embodiments of the processes comprising: (a) dissolving retigabine or a pharmaceutically acceptable salt thereof in a solvent to form a solution; (b) spraying the solution onto an inorganic carrier; and (c) optionally, drying the premix.

In embodiments of premixes of the present application, weight ratios of retigabine to inorganic carrier are in a range of about 10:90 to about 90:10.

In the present application, the average particle size of a premix composition comprising at least one inorganic carrier is generally from about 1 to about 1,000 μm.

Useful inorganic carriers for manufacturing a premix include, but are not limited to: salts of sodium, potassium, magnesium, and calcium, etc; and inorganic adsorbents such as amorphous forms of magnesium aluminometasilicate (e.g., Neusilin®), dibasic calcium phosphate anhydrous (e.g., Fujicalin®), hydrated aluminium silicate (kaolin), colloidal hydrated aluminium silicate (bentonite), calcium silicate (e.g., Zeopharm™ 600), attapulgite (purified native hydrated magnesium aluminum silicate), hydrous magnesium aluminum silicate (saponite), hectorite, aluminium oxide, and the like.

Specific examples of basic inorganic salts of sodium are sodium carbonate, sodium hydrogen carbonate, sodium hydroxide, and the like. Examples of basic inorganic salts of potassium are potassium carbonate, potassium hydrogen carbonate, potassium hydroxide, and the like. Examples of basic inorganic salts of magnesium are heavy magnesium carbonate, magnesium carbonate, magnesium oxide, magnesium hydroxide, magnesium silicate, magnesium aluminate, synthetic hydrotalcite [Mg₆Al₂(OH)₁₆.CO₃.4H₂O], aluminum hydroxide-magnesium oxide [2.5MgO.Al₂O₃.xH₂O], and the like. Examples of basic inorganic salts of calcium include precipitated calcium carbonate, calcium hydroxide, and the like.

Magnesium aluminometasilicate (Neusilin®) is a polymeric complex of magnesium, aluminum, silicon, oxygen, and water. It is a blend of colloidal montmorillonite and saponite that has been processed to remove grit and non-swellable ore components. Due to its properties such as improvement in flowability, providing high quality tablets from low compression forces, high specific surface area, high adsorption capacity, restricting reversion of amorphous form to crystalline state it can be used as an inorganic carrier for the present application.

Anhydrous dibasic calcium phosphate (Fujicalin®) is a stable inorganic compound used in pharmaceutical products because of its compaction properties, and the good flow properties of the coarse-grade material. The predominant deformation mechanism of anhydrous dibasic calcium phosphate coarse-grade is brittle fracture and this reduces the strain-rate sensitivity of the material, thus allowing easier transition from laboratory to production scale.

Solvents used in processes for manufacturing premixes include, but are not limited to, polyethylene glycols, glycerin, polyethylenes, glyceryl monostearate, glyceryl palmitostearate, polyvinyl alcohols, stearyl alcohol, stearic acid, waxes such as paraffin, spermaceti, anionic emulsifiers, nonionic emulsifiers, carnauba wax, cetyl esters, microcrystalline wax, white wax, yellow wax, oils such as hydrogenated castor oil and mineral oil, etc.

In embodiments, the application includes premixes for use in preparing pharmaceutical formulations of retigabine, premixes comprising in combination: (a) retigabine or a pharmaceutically acceptable salt thereof; and (b) magnesium aluminometasilicate.

In embodiments, the application includes premixes for use in preparing pharmaceutical formulations of retigabine, premixes comprising in combination: (a) retigabine or a pharmaceutically acceptable salt thereof; and (b) dibasic calcium phosphate anhydrous.

In embodiments, the application includes premixes for use in preparing pharmaceutical formulations of retigabine, premixes comprising in combination: (a) retigabine or a pharmaceutically acceptable salt thereof; and (b) calcium silicate.

In embodiments, the application includes premixes for use in preparing pharmaceutical formulations of retigabine, premixes comprising in combination: (a) retigabine or a pharmaceutically acceptable salt thereof; and (b) hydrated aluminum silicate (e.g., kaolin).

In embodiments, the application includes premixes for use in preparing pharmaceutical formulations of retigabine, premixes comprising in combination: (a) retigabine or a pharmaceutically acceptable salt thereof; and (b) colloidal hydrated aluminum silicates (e.g., bentonite).

The nature of retigabine in premixes of the application can be determined using analytical techniques such as X-ray powder diffraction (XRPD). Peaks attributable to a particular polymorphic crystalline form of retigabine will be seen in patterns obtained from samples of premixes. An XRPD pattern for an amorphous solid, solid solution, or dispersion generally does not contain sharp peaks, but may have a diffuse “halo” or broad hump. XPRD patterns of this application are generated using copper Kα radiation.

In aspects, the application includes methods for preparing retigabine premixes and pharmaceutical formulations containing the premixes.

In embodiments, premixes of retigabine of the present application are obtained by processes comprising: (a) adsorbing a solution of retigabine onto at least one pharmaceutically acceptable inorganic carrier; or (b) removing solvent from a solution or dispersion of retigabine and at least one pharmaceutically acceptable inorganic carrier; or (c) melt extruding a mixture of retigabine and at least one pharmaceutically acceptable inorganic carrier; or (d) grinding a mixture of retigabine and at least one pharmaceutically acceptable inorganic carrier; or (e) lyophilizing a mixture of retigabine and at least one pharmaceutically acceptable inorganic carrier; or (f) hot melting a mixture of retigabine and the at least one pharmaceutically acceptable inorganic carrier; optionally, in the presence of other excipients.

In embodiments, the application provides processes for preparing premixes for use in pharmaceutical formulations of retigabine, embodiments of processes comprising: (a) dissolving retigabine or a pharmaceutically acceptable salt thereof in a solvent; (b) adsorbing the solution onto an inorganic carrier; and (c) optionally, drying the premix.

In embodiments, premixes may be prepared by spray drying a suspension or solution of retigabine and an inorganic carrier. Alternatively, retigabine premixes may be prepared using fluid bed granulation techniques, where a solution of retigabine is sprayed onto an inorganic carrier.

Solid dispersions of the present application may be prepared using techniques known in the art, such as, for example, hot melting, solvent adsorption, and emulsion systems.

In embodiments, the application provides processes for preparing premixes for use in pharmaceutical formulations of retigabine, embodiments of processes comprising: (a) dissolving retigabine or a pharmaceutically acceptable salt thereof in a solvent; (b) mixing the resulting solution with inorganic carrier; and (c) optionally, drying the premix.

In embodiments, the application provides processes for preparing premixes for use in pharmaceutical formulations of retigabine, embodiments of processes comprising: (a) dissolving retigabine or a pharmaceutically acceptable salt thereof in a solvent; (b) spraying the solution onto an inorganic carrier; and (c) optionally, drying the premix.

Equipment suitable for processing pharmaceutical compositions of the present application include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans, tray dryers, fluid bed dryers, rotary cone vacuum dryers, and the like, multi-mills, fluid energy mills, ball mills, colloid mills, roller mills, hammer mills, and the like, equipped with a suitable screen.

In embodiments, the application provides processes for producing stable premixes, wherein the water content of a premix as determined using a Karl Fischer technique is about 0.5-10% by weight.

In embodiments, the application provides processes for producing stable premixes and/or compositions, wherein a premix has a loss on drying at 105° C. in the range of about 0.25-10%, or about 0.5-5%, by weight.

In embodiments, the present application provides pharmaceutical formulations containing a retigabine premix together with any one or more of pharmaceutically acceptable excipients such as diluents, binders, chelating agents, disintegrants, glidants, lubricants, anti-adherents. preservatives, colorants, sweeteners, plasticizers, film coating agents, and others. The premixes can be formulated into various pharmaceutical formulations such as powders, granules, capsules, tablets, pellets, etc.

In embodiments, stable retigabine premixes of the present application are in the form of powders, granules, pellets, spheroids, extrudates, and the like.

In embodiments, stable retigabine compositions of the present application are in the form of powders, made into a unit dosage form such as tablets.

In embodiments, stable retigabine compositions of the present application are in the form of powders, made into a unit dosage form such as capsules.

Various useful fillers or diluents for use in the present application include, but are not limited to, starches, lactose, cellulose derivatives, confectioner's sugar, and the like. Different grades of lactose include, but are not limited to, lactose monohydrate, lactose DT (direct tableting), lactose anhydrous, Flowlac™ (available from Meggle Products), Pharmatose™ (available from DMV), and others. Different starches include, but are not limited to, maize starch, potato starch, rice starch, wheat starch, pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products), and others. Various cellulose compounds that can be used include crystalline cellulose and powdered cellulose. Examples of crystalline cellulose products include, but are not limited to, Ceolus™ KG801, Avicel™ PH101, PH102, PH301, PH302 and PH-F20, PH-112 microcrystalline cellulose 114, and microcrystalline cellulose 112. Other useful diluents include, but are not limited to, carmellose, sugar alcohols such as mannitol (e.g., Pearlitol™ SD200), sorbitol, and xylitol, calcium carbonate, magnesium carbonate, dibasic calcium phosphate, and tribasic calcium phosphate.

Various useful binders according to the present application include, but are not limited to, hydroxypropylcelluloses, also called HPC (e.g., Klucel™ LF, Klucel™ EXF) and useful in various grades, hydroxypropyl methylcelluloses, also called hypromelloses or HPMC (e.g., Methocel™ products) and useful in various grades, polyvinylpyrrolidones or povidones (such as grades PVP-K25, PVP-K29, PVP-K30, and PVP-K90), copovidones, (e.g., Plasdone™ S 630), powdered acacia, gelatin, guar gum, carbomers (e.g., Carbopol® products), methylcelluloses, polymethacrylates, starches, and mixtures thereof.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (e.g., Ac-di-sol™ from FMC-Asahi Chemical Industry Co., Ltd.), crospovidones, examples of commercially available crospovidone products including, but not limited to, crosslinked povidone, Kollidon™ CL from BASF (Germany), Polyplasdone™ XL, XI-10, and INF-10 from ISP Inc. (USA), and low-substituted hydroxypropylcelluloses. Examples of low-substituted hydroxypropylcelluloses include, but are not limited to, low-substituted hydroxypropylcellulose LH11, LH21, LH31, LH22, LH32, LH2O, LH30, LH32 and LH33 (all manufactured by Shin-Etsu Chemical Co., Ltd.). Other useful disintegrants include sodium starch glycolate, colloidal (or fumed) silicon dioxide, starches and mixtures thereof.

Useful surface-active agents according to the present application include non-ionic, cationic or anionic or zwitterionic surface-active agents. Useful non-ionic surface-active agents include ethylene glycol stearates, propylene glycol stearates, diethylene glycol stearates, glycerol stearates, sorbitan esters (e.g., Span™ products) and polyhydroxyethylenically treated sorbitan esters (e.g., Tween™ products), aliphatic alcohols and PEG ethers, phenol and PEG ethers. Useful cationic surface-active agents include quaternary ammonium salts (e.g., cetyltrimethylammonium bromide) and amine salts (e.g., octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as, for example, soybean lecithin, egg yolk, and mixtures thereof.

Among the antioxidants, non-limiting examples include ascorbic acid and its salts, tocopherols, and sulfite salts, such as sodium metabisulfite or sodium sulfite, sodium sulfide, dl-alpha-tocopherol, butylated hydroxyanisole, butylated hydroxytoluene, ascorbyl palmitate, and propyl gallate. Other suitable antioxidants will be readily recognized by those skilled in the art.

Useful lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and combinations thereof.

One or more glidant materials, which improve the flow of powder blends, pellets, and mini-tablets, and minimize dosage form weight variations, can be used. Useful glidants include, but are not limited to, silicon dioxide, talc, and combinations thereof.

Colouring agents can be used to colour code the compositions, for example, to indicate the type and dosage of the therapeutic agent therein. Colouring agents can also be used to differentiate the varied fractions of multi-particulates contained in a unit dosage form such as a capsule. Suitable colouring agents include, without limitation, natural and/or artificial compounds such as FD&C colouring agents, natural juice concentrates, pigments such as titanium oxide, silicon dioxide, iron oxides, zinc oxide, combinations thereof, and the like.

Various solvents can be used in the processes of preparation of pharmaceutical compositions of the present application including, but not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, dimethylformamide, tetrahydrofuran, and any mixtures of two or more thereof.

pH-independent polymers that are useful in the present application include, but are not limited to, carbomers, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidones, polyvinyl acetates, polyvinyl alcohols, polyglycolides, polysiloxanes, polyurethanes and copolymers thereof, alkyl celluloses, hydroxyalkyl celluloses, cellulose ethers, cellulose esters, nitrocelluloses, methyl celluloses, ethyl celluloses, hydroxypropyl celluloses, hydroxypropyl methylcelluloses, hydroxybutyl methyl celluloses, natural polymers such as alginates and other polysaccharides that include but are not limited to arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starch, and various other natural homopolymer or heteropolymers such as those containing one or more of aldoses, ketoses, acids or amines, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof, and including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, synthetic polymers such as polymers of lactic acid and glycolic acid, polyanhydrides, poly (ortho) esters, polyurethanes, poly (butyric acid), poly (valeric acid), poly (caprolactone), poly (hydroxybutyrate), poly (lactide-co-glycolide) and poly (lactide-co-caprolactone) copolymers and mixtures thereof.

Various pH-dependent polymers that are useful in the present application include, but are not limited to, polymers and copolymers of acrylic and methacrylic acids, cellulose acetate butyrates, cellulose acetate phthalates, hydroxypropyl methyl cellulose phthalates, poly(methyl methacrylate), poly(ethylmethacrylate), poly(butylmethacrylate), poly(isobutylmethacrylate), poly(hexlmethacrylate), poly(isodecylmethacrylate), poly(laurylmethacrylate), poly(phenyl methacrylate), poly(methyl acrylate), poly(isopropyl acrylate), poly(isobutyl acrylate), poly(octadecyl acrylate), and any mixtures thereof.

In embodiments, one or more pH-independent or pH-dependent polymers are used for coating formulations of the present application.

Other useful additives for coatings include, but are not limited to, plasticizers, antiadherents, opacifiers, solvents, and optionally colorants, lubricants, pigments, antifoam agents, and polishing agents.

Various useful plasticizers include, but are not limited to, substances such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent.

Antiadhesives are frequently used in film coating processes to avoid sticking effects during film formation and drying. An example of a useful antiadhesive for this purpose is talc. The antiadhesive is frequently present in the film coating in an amount of about 0.5% to 15%, based upon the total weight of the coating.

The foregoing descriptions of excipients are not intended to be exhaustive. Those skilled in the art will be aware of many other substances that are useful in the practice of the application, and the use of such substances is specifically included in this application.

The different physicochemical properties of the active ingredient and as well as of excipients are to be considered, as these properties affect the process and formulation properties. Various important physicochemical properties include but are not limited to particle size, density (bulk density and tapped density), compressibility index, Hausner's ratio, angle of repose, etc. Particle sizes of active pharmaceutical ingredient can affect the solid dosage form in numerous ways. For example, drug content uniformity of pharmaceutical dosage units can be affected by particle sizes and size distribution. This will be even more critical for low-dose drugs and satisfactory dosage units of low doses cannot be manufactured from a drug that does not meet certain particle size and size distribution criteria. Also particle size plays an important role in dissolution of active ingredient form the final dosage form for certain drugs like retigabine because of their low solubility. Hence, these physicochemical properties not only affect the process of the preparing the pharmaceutical compositions but also affect the performance of the pharmaceutical product both in vitro and in vivo.

The physicochemical properties of the retigabine premixes of the application can be readily controlled through the choice of an appropriate pharmaceutically acceptable carrier that is used in premix preparation. Thus, for example, the particle sizes and distribution of the retigabine premixes of the application can be readily controlled by the proper choice of the pharmaceutically acceptable carrier with defined particle sizes and distribution. Thus, if a larger particle size premix is required, a carrier having large particles should be appropriately chosen and, vice versa, if a smaller particle size premix is desired. The selection of appropriate particle sizes of retigabine as well as of carrier is within the scope of the application. Mixing of more than one particle size carrier species is also within the scope of the application. Also, included are mixtures of premixes of retigabine wherein the carriers that are used in premix preparation are different.

The D10, D50, and D90 values are useful ways for indicating a particle size distribution. D90 is the size value where at least 90 volume percent of the particles have a size smaller than the said value. Likewise D10 refers to 10 volume percent of the particles having a size smaller than the said value. D50 refers to at least 50 volume percent of the particles having a size smaller than the said value and D[4,3] value refers to the mean particle size. Methods for determining D10, D50 D90 and D[4,3] include laser light diffraction, such as using equipment sold by Malvern Instruments Ltd., Malvern, Worcestershire, United Kingdom. Other types of equipment may be used, as is known in the art.

Flowability of materials can be measured and represented using the Carr Index. The Carr Index is the percent ratio of the difference between tapped density and bulk density to tapped density, mathematically described as:

Carr Index=[(Tapped density−Bulk density)+Tapped density]×100.

The densities can be determined using the standard test method 616 “Bulk Density and Tapped Density” in United States Pharmacopeia 29, United States Pharmacopeial Convention, Inc., Rockville, Md., 2005.

Carr Index values below about 15% represent materials with very good flow properties and values above about 40% represent materials with very poor flow properties. The retigabine premixes of the present application typically have a Carr Index which is substantially lower than the 40% described for products with poor flow properties. Values for Carr Index for the retigabine premixes of the application are generally less than about 35%, or less than about 30%, or less than about 25%, or less than about 20%, or less than about 15%. This indicates superior handling capabilities during processing into pharmaceutical dosage forms. In embodiments, premixes of the present application have Carr index values in the range of about 1-40%.

Retigabine premixes of the present application can be further processed into various pharmaceutical dosage forms as prepared, or can be combined with one or more pharmaceutically acceptable excipients. The different pharmaceutical dosage forms where the retigabine premixes of the application find utility include: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release, controlled release or their combinations. Further, immediate release formulations may be conventional, dispersible, chewable, mouth dissolving, or flash melt preparations. Delayed release or controlled release formulations may comprise hydrophilic, lipophilic, or hydrophobic release rate controlling substances, or their combinations to form matrix or reservoir, or combinations of matrix and reservoir systems. The formulations may be prepared using any of direct blending, dry granulation, wet granulation, or extrusion and spheronization. Formulations may be presented as uncoated, film coated, sugar coated, powder coated, and enteric coated or modified release forms. Formulations of the present application may further comprise one or more pharmaceutically acceptable excipients.

One or more polymers that can be used in present application for modified release include hydrophilic, hydrophobic and lipophilic substances, and combinations thereof.

Examples of the suitable agents include, but are not limited to, hydroxypropyl methylcelluloses, hydroxypropyl celluloses, and the like. Film coatings are not limited to water-soluble film coatings. If necessary, a gastric coating or an enteric coating may be applied to give a pharmaceutical formulation intended to rapidly release the pharmaceutically active ingredient following dissolution of the coating film. Alternatively, it is also acceptable not to apply a film coating.

The term “immediate release” as used herein means a pharmaceutical formulation, from which the active ingredient is immediately released upon administration to a subject.

The term “modified release pharmaceutical formulation” as used herein means a pharmaceutical formulation such that the active ingredient is delivered over a sustained period of time, following administration to a subject, based on delaying the rate of release and/or the onset of release of the active ingredient.

As used herein the terms “delayed release” or “enteric coated” means the release of the active substance is modified to occur at a later time than that from an immediate release form, following administration.

A coating can be applied to premix particles or formulations thereof, such as tablets, by conventional coating procedures in a suitable coating pan or in fluidized bed apparatus, using water and/or an organic solvent for the coating solutions or dispersions. Water soluble or insoluble polymers that can be used for an inert coating include, for example, sugars, zein, hydroxypropyl celluloses, hydroxypropyl methylcelluloses, hydroxyethyl celluloses, polyvinylalcohols, providones, polyethylene glycols, poloxamers, ethylcelluloses, gelatin, polylysine, polyarginine, polyglycine, polyvinylpyrrolidines, vinyl acetate copolymers, and mixtures of any two or more thereof.

In the case of tablets, coatings may also be applied using a dry coating technique. A coating may also include pharmaceutically acceptable water-soluble or tablet excipients that rapidly dissolve or disintegrate in water. Ordinary plasticizers, pigments, titanium dioxide, talc and other additives may also be included in a coating. In the case of gelatin capsules, the gelatin capsule itself serves as a coating, although capsules can also be coated with films. The quantity of a coating of the present application may vary from about 0.3% to 6%, or about 0.5 to 4%, or about 1-3%, of the total weight of a core.

An enteric coating can be applied either directly onto a core by conventional coating techniques such as, for instance, pan coating or fluidized bed coating using solutions of polymers in water and/or suitable organic solvents, or by using latex suspensions of polymers. Enteric coating polymers that can be used, for example, include cellulose acetate phthalates (CAP), hydroxypropyl methylcellulose phthalates (HPMCP), polyvinyl acetate phthalates (PVAP), hydroxypropyl methylcellulose acetate succinates (HPMCAS), cellulose acetate trimellitates, hydroxypropyl methylcellulose succinates, cellulose acetate succinates, cellulose acetate hexahydrophthalates, cellulose propionate phthalates, copolymers of methylmethacrylic acid and methyl methacrylate, copolymers of methyl acrylate, methylmethacrylate and methacrylic acid, copolymers of methylvinyl ether and maleic anhydride (Gantrez™ ES series), ethyl methyacrylate-methylmethacrylate-chlorotrimethylammonium ethyl acrylate copolymers, natural resins such as zein, shellac and copal collophorium, carboxymethyl ethylcelluloses, co-polymerized methacrylic acid/methacrylic acid methyl esters such as, for instance, materials sold under the trade name Eudragit® L12.5, L100, or Eudragit® S12.5, S100, and several commercially available enteric dispersion systems (e.g., Eudragit® L30D55, Eudragit® FS30D, Eudragit® L100-55, Eudragit® S100 (Evonik Industries, Germany), Kollicoat® MAE30D and 30DP (BASF), Estacryl® 30D (Eastman Chemical), Aquateric® and Aquacoat® CPD30 (FMC), and mixtures thereof.

The enteric coating layer can optionally contain a pharmaceutically acceptable plasticizer such as, for instance, cetanol, triacetin, citric acid esters such as, for instance, those known under the trade name Citroflex® (from Pfizer, New York), phthalic acid esters, dibutyl succinate or similar plasticizers. The amount of plasticizer is usually optimized for each enteric coating polymer and is usually in the range of about 1-20% of the enteric coating polymer. Dispersants such as talc, colorants and pigments may also be included into the enteric coating layer. The weight of enteric coating applied is usually about 1-12%, or about 2-10%, or about 4-8%, of the weight of core tablet.

In embodiments, the application includes packaging for the retigabine premixes and/or compositions, which maintains stability during storage and transportation. The stabilization of the retigabine premix and/or composition of the present application can be improved by using packaging inhibiting the permeation of oxygen and moisture, packaging having inert gases (namely, packages with air replaced with gases other than oxygen), vacuum packaging, and packages containing a deoxidizer. The stabilization is improved by reducing oxygen with which the solid preparation is directly brought in contact, using these package forms. When a deoxidizer is enclosed, the pharmaceutical solid preparation can be packed with an oxygen permeating material, and then this is enclosed within other packaging.

In embodiments, stable premixes and/or compositions of the present application include a desiccant and/or an oxygen absorbent as a component of packaging. A desiccant is a hygroscopic substance that induces or sustains a state of dryness (desiccation) in its local vicinity in a moderately well-sealed container. Commonly encountered pre-packaged desiccants are solids, and work through absorption or adsorption of water, or a combination of the two. Desiccants for specialized purposes may be in forms other than solid, and may work through other principles, such as chemical bonding of water molecules. Pre-packaged desiccants are most commonly used to remove excessive humidity that would normally degrade or even destroy products sensitive to moisture. Non-limiting examples of various desiccants are anhydrous calcium sulfate (e.g., Drierite® products), silica gel, calcium sulfate, calcium chloride, montmorillonite clay, and molecular sieves. Commercially available oxygen absorbant products such as StabilOx® are useful in minimizing the degradation of active agent due to oxidation.

In embodiments, the application includes the use of packaging materials such as containers and closures of high-density polyethylene (HDPE), low-density polyethylene (LDPE) and or polypropylene and/or glass, and blisters or strips composed of moisture resistant aluminum, high-density polypropylene, or polyvinyl chloride and/or polyvinylidene dichloride.

In embodiments, the application includes forms of packaging for compositions of retigabine, such that retardation of drug degradation from the compositions is prevented. In embodiments, the application provides a package suitable for commercial sale, which provides stability during storage, transportation, and use.

In embodiments, the pharmaceutical dosage forms of the present application are orally administered to a patient in need thereof.

In aspects, the application includes methods of treating patients suffering from conditions such as epilepsy as an adjunctive treatment of partial onset seizures with or without secondary generalisation in adults, using pharmaceutical formulations of the present application.

Certain specific aspects and embodiments will be more fully explained by the following examples. However, it should be understood that the scope of the present disclosure is not to be limited by the examples in any manner. Those skilled in the art will be aware that many modifications and alterations can be made, and all of these are within the technical scope of the present application.

### EXAMPLES

### Example 1

### Retigabine Premix

Crystalline retigabine (400 mg), having forms A and C in a weight ratio of 95:5, respectively, was dissolved in glycerin (2.3 mL) and stirred to form a clear solution. Magnesium aluminometasilicate (Neusilin®, 1200 mg) was added to the solution and mixed to obtain a premix.

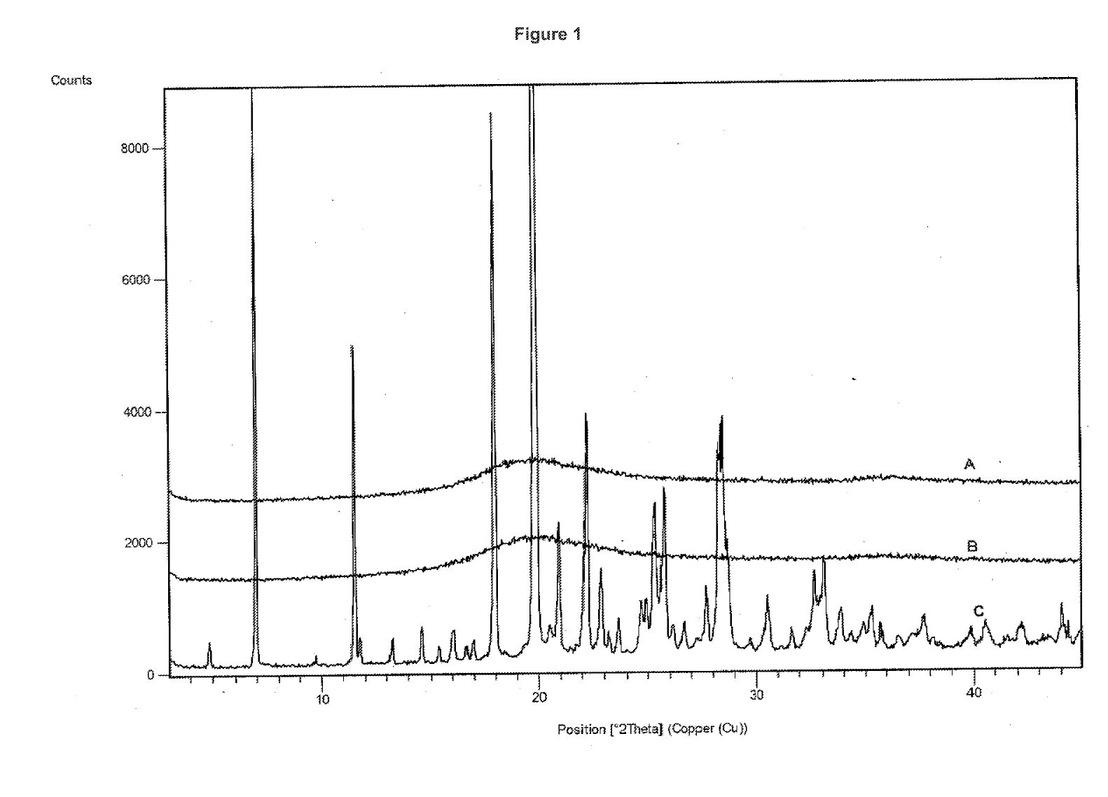

A sample of the premix was subjected to XRPD analysis. FIG. 1 represents the comparative XRPD patterns for the crystalline retigabine ingredient (A), the premix (B), and a placebo (C) prepared without the retigabine. Retigabine in the premix was observed to be present in an amorphous form.

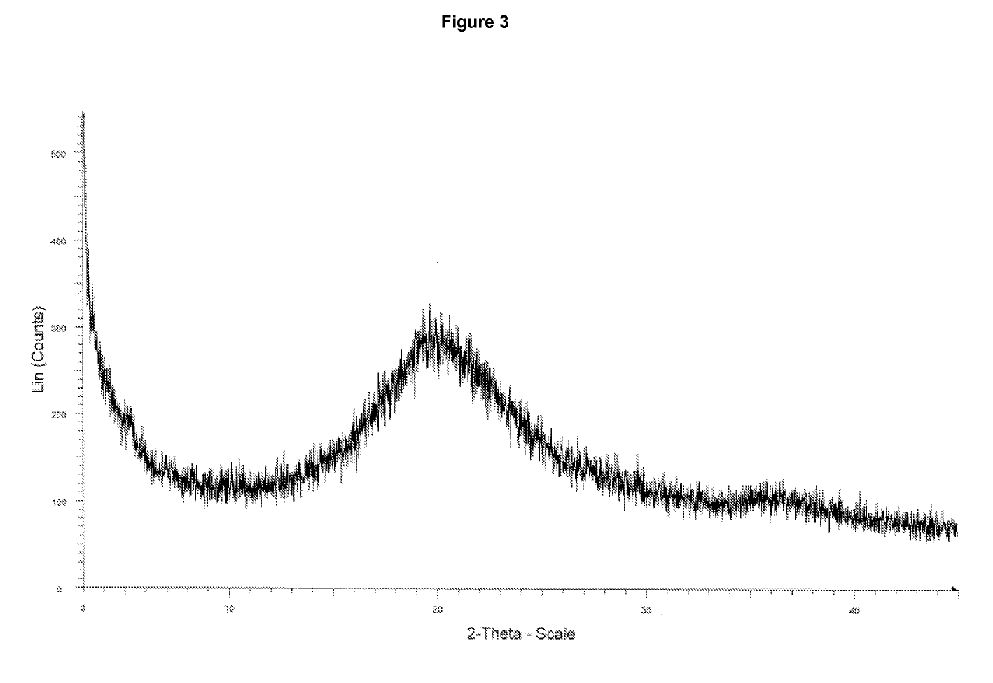

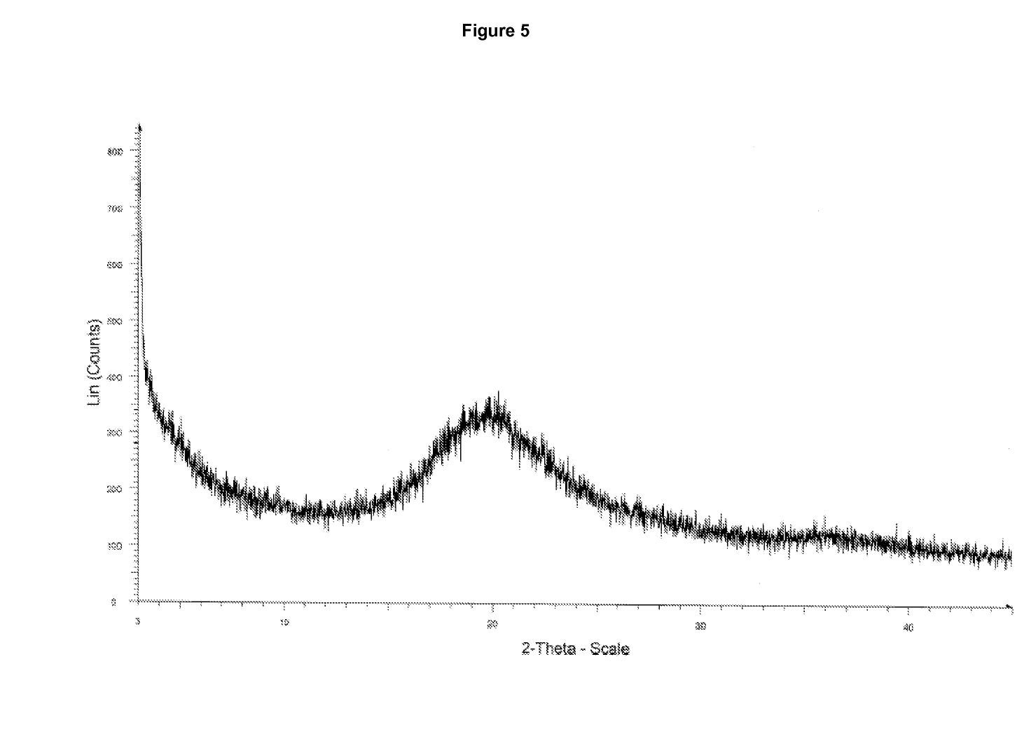

Premixes of Example 1 were packaged in a polyethylene pouch and placed in closed HDPE container and stored under 2 to 8° C. for 16 months. The samples were analyzed by XRPD. FIG. 3 shows patterns for the premix after storage at 2 to 8° C. for 16 months. Further, the premixes of Example 1 were packaged in a polyethylene pouch and placed in closed HDPE container and stored under 2 to 8° C. for 16 months, followed by storage at 40° C. and 75% relative humidity for 1 week. FIG. 5 shows patterns for the premix after storage at 2 to 8° C. for 16 months, followed by storage at 40° C. and 75% relative humidity for 1 week. It was observed that retigabine retains its amorphous form in the premix during storage.

### Example 2

### Retigabine Premix

Crystalline retigabine (400 mg), having forms A and C in a weight ratio of 95:5, respectively, was dissolved in glycerin (1.8 mL) and stirred to form a clear solution. Dibasic calcium phosphate anhydrous (Fujicalin®, 2800 mg) was added to the solution and mixed to obtain a premix.

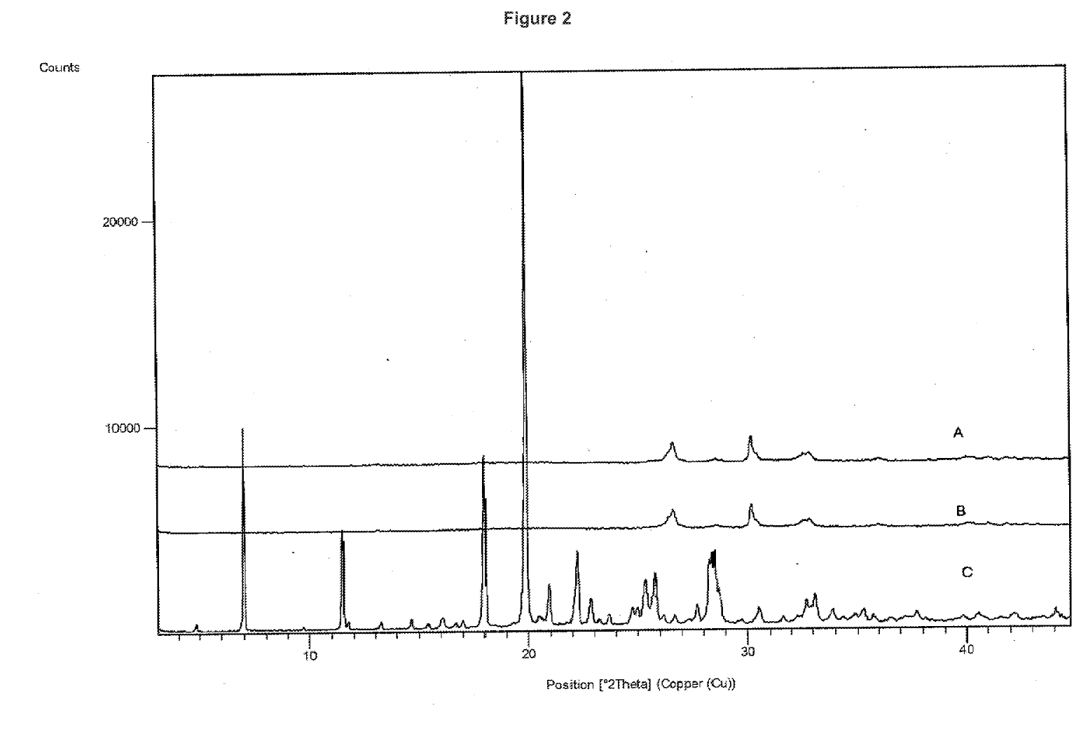

A sample of the premix was subjected to XRPD analysis. FIG. 2 represents the comparative XRPD patterns for the crystalline retigabine ingredient (A), the premix (B), and a placebo (C) prepared without the retigabine. Retigabine in the premix was observed to be present in an amorphous form.

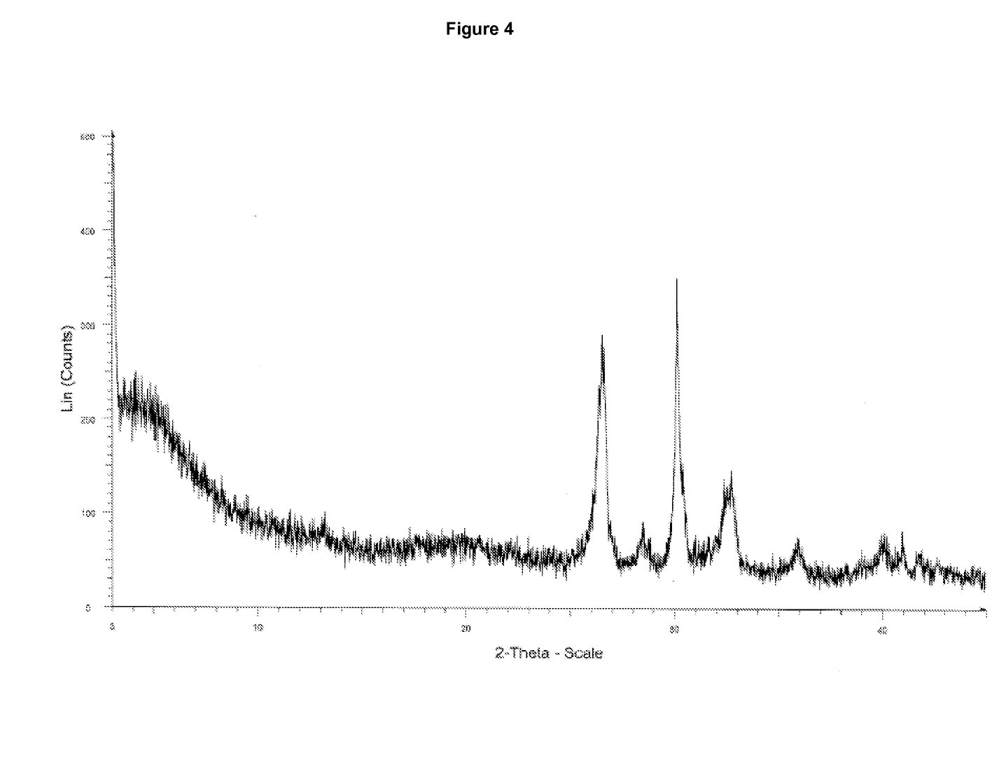

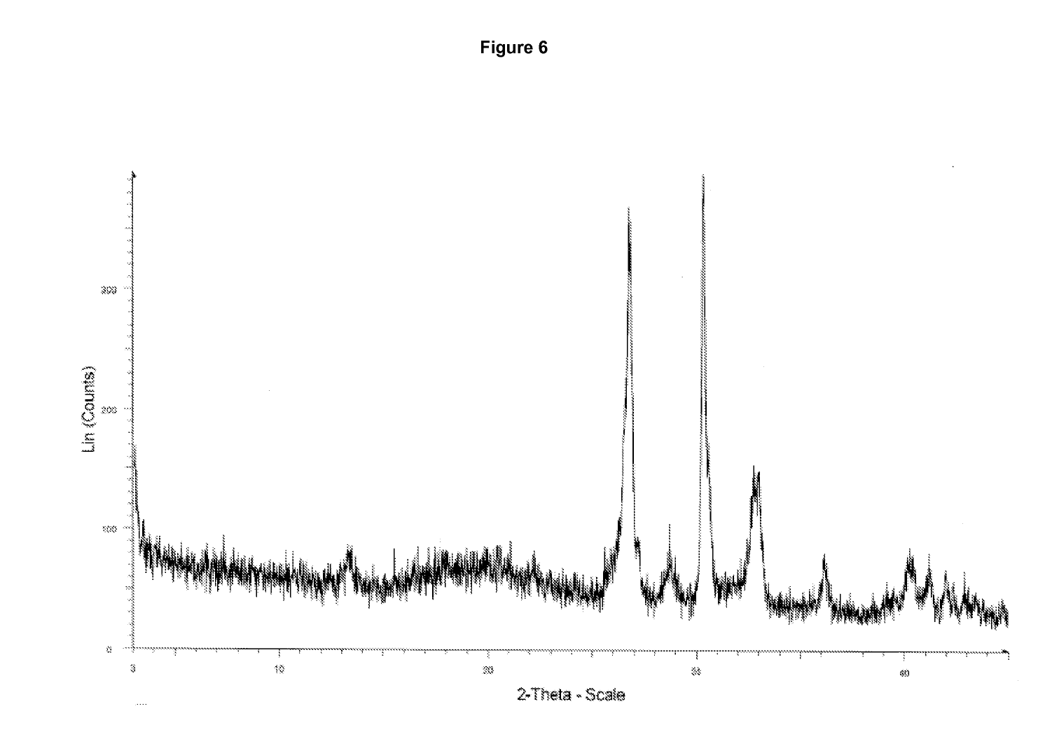

Premixes of Example 2 were packaged in a polyethylene pouch and placed in closed HDPE container and stored under 2 to 8° C. for 16 months. The samples were analyzed by XRPD. FIG. 4 shows patterns for the premix after storage at 2 to 8° C. for 16 months. Further, the premixes of Example 2 were packaged in a polyethylene pouch and placed in closed HDPE container and stored under 2 to 8° C. for 16 months, followed by storage at 40° C. and 75% relative humidity for 1 week. FIG. 6 shows patterns for the premix after storage at 2 to 8° C. for 16 months, followed by storage at 40° C. and 75% relative humidity for 1 week. It was observed that retigabine retains its amorphous form in the premix during storage.

### Example 3

### Retigabine Premix

Retigabine (400 g) is dissolved in a 1:1 mixture by volume of isopropyl alcohol and methanol (300 mL) and stirred to form a clear solution. The solution is sprayed onto Fujicalin (1200 g) in a fluidised bed processor, to obtain a premix.

### Example 4

### Retigabine Premixes

| | Quantity | | | |
|---|---|---|---|---|
| Ingredient | 4A | | 4B | |
| Retigabine crystalline | 400 | g | 400 | g |

| | | | |
|---|---|---|---|
| Polyethylene glycol | 300 | mL | — |

| | | | |
|---|---|---|---|
| Propylene glycol | — | 300 | mL |

| | | | |
|---|---|---|---|
| Hydrated aluminum silicate (kaolin) | 2500 | g | — |

| | | | |
|---|---|---|---|
| Colloidal hydrated aluminum silicate (bentonite) | — | 2500 | g |

Manufacturing procedure for 4A:
1. Dissolve retigabine in polyethylene glycol and stir to form a clear solution.
2. Spray the solution onto kaolin in a fluidised bed processor, to obtain a premix.

Manufacturing procedure for 4B:
1. Dissolve retigabine in polypropylene glycol and stir to form a clear solution.
2. Mix the solution with bentonite in a double cone blender, to obtain a premix.

### Example 5

### Retigabine 100 mg Tablets

| Ingredient | mg per Tablet |
|---|---|
| Retigabine* | 100 |
| Lactose | 260 |
| Hydroxypropyl cellulose | 12 |
| Starch | 20 |
| Magnesium stearate | 4 |
| Colloidal silica (Aerosil ®) | 4 |

*Retigabine contained in an appropriate quantity of a premix prepared according to Example 1.

Manufacturing procedure:
1. Blend retigabine premix with lactose.
2. Granulate the blend with a solution of hydroxypropyl cellulose in water, using a fluidized bed processor, and dry the granules.
3. Mill the granules, then blend with starch.
4. Add magnesium stearate and blend, then add Aerosil and blend.
5. Compress the blend into tablets.

### Example 6

### Retigabine 100 mg Tablets

| Ingredient | mg per Tablet |
|---|---|
| Retigabine* | 100 |
| Microcrystalline cellulose | 130 |
| Lactose spray dried (Flowlac ®) | 130 |
| Polyvinylpyrrolidone | 8 |
| Croscarmellose sodium | 20 |
| Magnesium stearate | 8 |
| Aerosil | 4 |

*Retigabine contained in an appropriate quantity of a premix prepared according to Example 2.

Manufacturing procedure:
1. Blend retigabine premix with microcrystalline cellulose and lactose.
2. Add polyvinylpyrrolidone and croscarmellose sodium, and blend.
3. Add magnesium stearate and blend, then add Aerosil and blend.
4. Compress the blend into tablets.

### Example 7

### Retigabine 200 mg Tablets

| Ingredient | mg per Tablet |
|---|---|
| Retigabine* | 200 |
| Lactose compressible (Ludipress ®) | 110 |
| Hydroxypropyl methylcellulose, 3 cps | 12 |
| Croscarmellose sodium | 20 |
| Magnesium stearate | 4 |
| Aerosil | 4 |

*Retigabine contained in an appropriate quantity of a premix prepared according to Example 1.

Manufacturing procedure:
1. Blend retigabine premix with lactose.
2. Add hydroxypropyl methylcellulose and croscarmellose sodium, then blend.
3. Add magnesium stearate and blend, then add Aerosil and blend.
4. Compress the blend into tablets.

### Example 8

### Retigabine 50 mg Tablets

| Ingredient | mg per Tablet |
|---|---|
| Retigabine | 50 |
| Mannitol | 145 |
| Cellulose powdered | 145 |
| Polyvinylpyrrolidone (PVP K30) | 19.5 |
| Crospovidone | 20 |
| Magnesium stearate | 4 |
| Aerosil | 4 |

* Retigabine contained in an appropriate quantity of a premix prepared according to Example 4A.

Manufacturing procedure:
1. Blend retigabine premix with mannitol, powdered cellulose, and PVP K30.
2. Add crospovidone and blend.
3. Compact the blend, then mill to obtain granules.
4. Add magnesium stearate and blend, then add Aerosil and blend.
5. Compress the blend into tablets.

### Example 9

### Retigabine 100 mg Capsules

| Ingredient | mg per Tablet |
|---|---|
| Retigabine* | 100 |
| Mannitol | 145 |
| Powdered cellulose | 145 |
| Magnesium stearate | 5 |
| Aerosil | 5 |

*Retigabine contained in an appropriate quantity of a premix prepared according to Example 1.

Manufacturing procedure:
1. Blend retigabine premix with mannitol and powdered cellulose.
2. Add magnesium stearate and blend, then add Aerosil and blend.
3. Fill the blend into hard gelatin capsules.

1. A stable premix comprising retigabine, or a pharmaceutically acceptable salt thereof, and an inorganic carrier.

2. The stable premix according to claim 1, wherein the inorganic carrier comprises one or more of magnesium aluminometasilicate, dibasic calcium phosphate anhydrous, hydrated aluminium silicate, colloidal hydrated aluminium silicate, calcium silicate, hydrated magnesium aluminum silicate, purified native hydrated magnesium aluminum silicate, hectorite, aluminium oxide, sodium carbonate, sodium hydrogen carbonate, sodium hydroxide, potassium carbonate, potassium hydrogen carbonate, potassium hydroxide, magnesium carbonate, magnesium oxide, magnesium hydroxide, magnesium silicate, magnesium aluminate, synthetic hydrotalcite, aluminum hydroxide-magnesium oxide, precipitated calcium carbonate, and calcium hydroxide.

3. The stable premix according to claim 1, wherein weight ratio of the retigabine to the inorganic carrier are in the range of about 10:90 to about 90:10.

4. The stable premix according to claim 1, wherein the retigabine in the premix is in amorphous form.

5. The stable premix according to claim 1, wherein the retigabine in the premix is in the form of a solid dispersion.

6. The stable premix according to claim 1, wherein the retigabine in the premix is in the form of a solid solution.

7. A process for preparing the stable premix according to claim 1, the process comprising:
(a) dissolving retigabine or a pharmaceutically acceptable salt thereof in a solvent to form a solution;
(b) adsorbing the solution onto the inorganic carrier; and
(c) optionally, drying the premix.

8. The process according to claim 7, wherein the adsorbtion of the solution onto the inorganic carrier comprises mixing the solution with inorganic carrier.

9. The process according to claim 7, wherein the adsorbtion of the solution onto the inorganic carrier comprises spraying the solution onto the inorganic carrier.

10. The process according to claim 7, wherein the solvent comprises one or more of polyethylene glycols, glycerin, polyethylenes, glyceryl monostearate, glyceryl palmitostearate, polyvinyl alcohols, stearyl alcohol, stearic acid, waxes such as paraffin, spermaceti, anionic emulsifiers, nonionic emulsifiers, carnauba wax, cetyl esters, microcrystalline wax, white wax, yellow wax, oils such as hydrogenated castor oil, and mineral oil.

11. A pharmaceutical composition, comprising the stable premix according to claim 1, and at least one pharmaceutically acceptable excipient.

12. The pharmaceutical composition according to claim 11, in the form of tablets, caplets, capsules, oral disintegrating dosage forms, chewable dosage forms, pills, granules, and sachets.

13. The pharmaceutical composition according to claim 11, wherein pharmaceutically acceptable excipients are one or more of diluents, binders, chelating agents, disintegrants, glidants, lubricants, anti-adherents, preservatives, colorants, sweeteners, plasticizers, and film coating agents.

14. A method of treating epilepsy in a mammal, comprising administering to the mammal an effective amount of the pharmaceutical composition according to claim 11.
